(19) United States
(12) Patent Application Publication
(10) Pub. No.: US 2025/0209625 A1
(43) Pub. Date: Jun. 26, 2025

(54) MEDICAL IMAGE PROCESSING APPARATUS, MEDICAL IMAGE PROCESSING METHOD, AND NON-TRANSITORY COMPUTER-READABLE STORAGE MEDIUM STORING MEDICAL IMAGE PROCESSING PROGRAM

(71) Applicant: CANON MEDICAL SYSTEMS CORPORATION, Otawara-shi (JP)

(72) Inventors: Shuhei BANNAE, Utsunomiya (JP); Hiroshizu MORISHIMA, Utsunomiya (JP)

(73) Assignee: CANON MEDICAL SYSTEMS CORPORATION, Otawara-shi (JP)

(21) Appl. No.: 18/986,365

(22) Filed: Dec. 18, 2024

(30) Foreign Application Priority Data
Dec. 20, 2023 (JP) 2023-214791

(51) Int. Cl. G06T 7/00; A61B 5/02; A61B 5/055; G06T 7/68

(52) U.S. Cl. CPC G06T 7/0014; A61B 5/02007; A61B 5/055; G06T 7/68; G06T 2207/30016; G06T 2207/30096

(57) ABSTRACT

A medical image processing apparatus includes processing circuitry that identifies a first area with a lesion in a site of a subject based on a first medical image generated by first imaging of the site; determines whether the first area is bilateral or unilateral relative to the site, based on a position of the first area; upon determining that as bilateral, decides, as a second area, a normal portion in the site in a second medical image generated by different, second imaging; upon determining that as unilateral, decides, in the second medical image, as the second area, an area line symmetric to the first area about a centerline of the site; calculates, in the second medical image, first and second feature amounts of the respective first and second areas; and calculates an index representing mismatch between the first and second medical images, based on the first and second feature amounts.

### CROSS-REFERENCE TO RELATED APPLICATIONS

This application is based upon and claims the benefit of priority from Japanese Patent Application No. 2023-214791, filed on Dec. 20, 2023, the entire contents of which are incorporated herein by reference.

### FIELD

Embodiments described herein relate generally to a medical image processing apparatus, a medical image processing method, and a non-transitory computer-readable storage medium storing a medical image processing program.

### BACKGROUND

Conventionally, as a method of predicting an onset time of cerebral infarction, there is a method of focusing on a mismatch between a diffusion weighted image (DWI) and a fluid attenuated inversion recovery (FLAIR) image (DWI/FLAIR mismatch phenomenon) among magnetic resonance imaging (MRI) images.

The DWI image can depict an acute phase cerebral infarction. On the other hand, there is no change found in the FLAIR image in a case of an acute phase cerebral infarction. Therefore, as illustrated in FIG. 10, a user determines whether or not the cerebral infarction of a subject is a cerebral infarction in the acute phase by using a difference (mismatch) between the DWI image and the FLAIR image. As illustrated in FIG. 10, in many cases, a case where there is a mismatch between the DWI image and the FLAIR image (TMM) corresponds to a cerebral infarction in an acute phase, and a case where there is no mismatch (NMM) corresponds to a cerebral infarction in a non-acute phase.

In the case of acute phase cerebral infarction, for example, an effect of removing thrombus by administration of recombinant tissue-type plasminogen activator (rt-PA) is observed. For this reason, there is a demand for understanding whether or not a cerebral infarction that has occurred in a subject is in the acute phase. A cerebral infarction often occurs from midnight to the morning, for example, during sleep, and an onset time of cerebral infarction is often unknown. Therefore, by focusing on a DWI/FLAIR mismatch phenomenon, it may be possible to understand an approximate elapsed time from the onset of the cerebral infarction.

In the DWI/FLAIR mismatch phenomenon, first, an area on an affected side (hereinafter, referred to as an affected side area) of the area related to the cerebral infarction is identified in the DWI image, and the affected side area is mapped onto the FLAIR image. Next, in the FLAIR image, an area corresponding to line symmetry of the affected side area with the center line of the brain as a symmetry axis is decided as an area on an unaffected side (hereinafter, referred to as an unaffected side area). Subsequently, it is known to quantify the DWI/FLAIR mismatch phenomenon by using the affected side area and the unaffected side area in the FLAIR image.

However, in a case where a subject has infarcts on both the left and right sides of the brain, such as cerebellar infarction or bilateral infarction, quantification of the known DWI/FLAIR mismatch phenomenon cannot distinguish between the affected side and the unaffected side. Thus, it is difficult to quantitatively evaluate the DWI/FLAIR mismatch phenomenon between the affected side area and the unaffected side area. Therefore, a doctor qualitatively determines the DWI/FLAIR mismatch phenomenon in his or her own mind.

### BRIEF DESCRIPTION OF THE DRAWINGS

FIG. 1 is a block diagram illustrating an example of a configuration of a medical information processing system including a medical image processing apparatus according to an exemplary embodiment;

FIG. 2 is a diagram illustrating an example of a first area and a symmetric area in a second medical image according to the exemplary embodiment;

FIG. 3 is a diagram illustrating another example of a first area and a symmetric area in a second medical image according to the exemplary embodiment;

FIG. 4 is a diagram illustrating yet another example of a first area and a symmetric area in a second medical image according to the exemplary embodiment;

FIG. 5 is a diagram illustrating an example of a first area and a second area in a case where the first area is bilateral according to the exemplary embodiment;

FIG. 6 is a flowchart illustrating an example of a procedure of mismatch evaluation processing according to the exemplary embodiment;

FIG. 7 is a diagram illustrating an example in which a first area and a second area are superimposed on a second medical image and displayed on a display together with a legend of mismatch indices, in a case where the first area is determined to be unilateral according to the exemplary embodiment;

FIG. 8 is a diagram illustrating an example in which a first area and a second area are superimposed on a second medical image and displayed on a display together with a legend of mismatch indices, in a case where the first area is determined to be bilateral according to the exemplary embodiment;

FIG. 9 is a diagram illustrating an example in which a plurality of segment areas is further superimposed in the display example illustrated in FIG. 8; and

FIG. 10 is a diagram illustrating an example of a difference (mismatch) between a DWI image and a FLAIR image according to the related art.

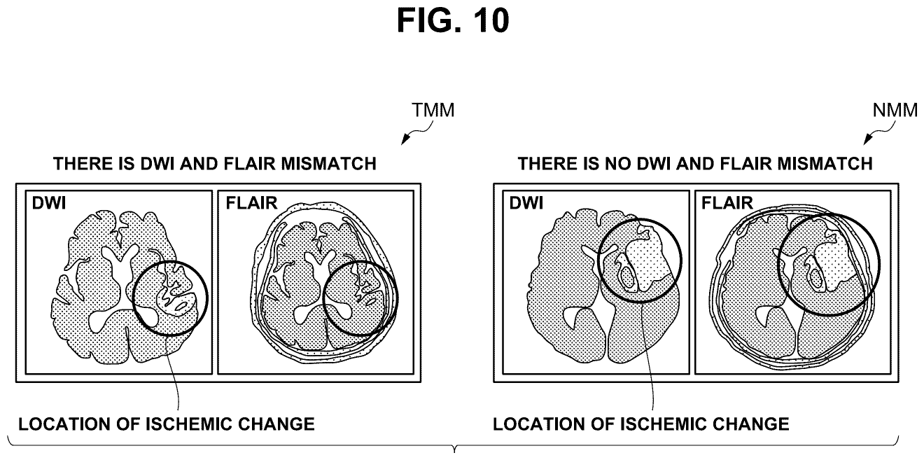

### DETAILED DESCRIPTION

A medical image processing apparatus according to an exemplary embodiment includes processing circuitry. The processing circuitry acquires a first medical image collected by predetermined imaging of an imaging site of a subject and a second medical image collected by imaging different from the predetermined imaging and including the imaging site, identifies a first area related to a lesion in the imaging site, based on the first medical image, determines whether the first area is bilateral or unilateral with respect to the imaging site, based on a position of the first area in the imaging site, decides an area different from the first area in the second medical image as a second area indicating a normal portion in the imaging site in a case where it is determined that the first area is bilateral, decides, as the second area, an area corresponding to line symmetry of the first area with a center line relative to the imaging site as a symmetry axis in the second medical image in a case where it is determined that the first area is unilateral, calculates, in the second medical image, a first feature amount indicating a feature based on a plurality of pixel values in the first area and a second feature amount indicating a feature based on a plurality of pixel values in the second area, and calculates an index related to a mismatch between the first medical image and the second medical image, based on the first feature amount and the second feature amount.

Various Embodiments will be described hereinafter with reference to the accompanying drawings.

Hereinafter, exemplary embodiments of a medical image processing apparatus, a medical image processing method, and a medical image processing program will be described with reference to the drawings. In the following exemplary embodiments, parts denoted by the same reference numeral perform the same operation, and redundant description will be omitted as appropriate.

### Exemplary Embodiment

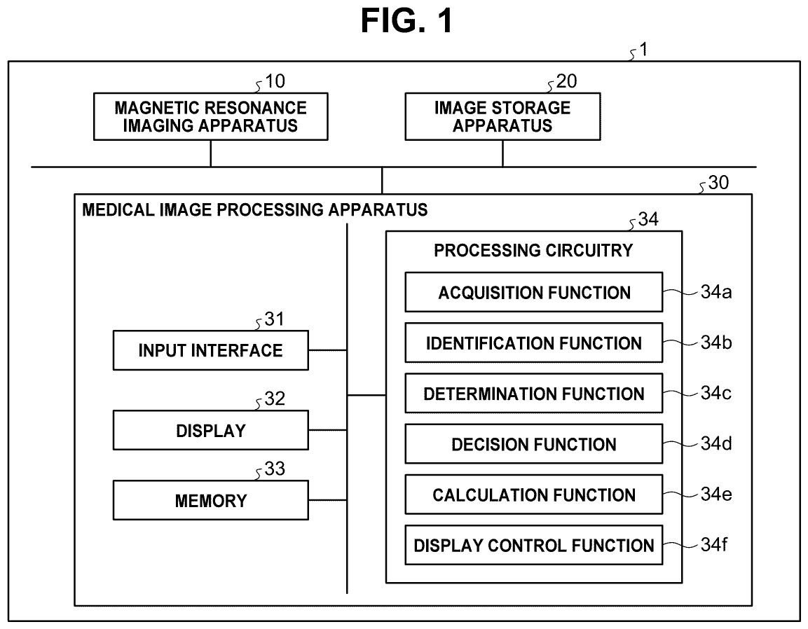

FIG. 1 is a block diagram illustrating an example of a configuration of a medical information processing system 1 including a medical image processing apparatus 30 according to an exemplary embodiment. As illustrated in FIG. 1, the medical information processing system 1 according to the exemplary embodiment includes a magnetic resonance imaging (MRI) apparatus 10, an image storage apparatus 20, and a medical image processing apparatus 30. As illustrated in FIG. 1, the MRI apparatus 10, the image storage apparatus 20, and the medical image processing apparatus 30 are connected to each other via a network.

The MRI apparatus 10 collects a magnetic resonance image (MR image) from a subject P. For example, the MRI apparatus 10 collects MR data from the subject P and reconstructs the MR data to generate an MR image. The MRI apparatus 10 transmits the generated MR image to the image storage apparatus 20 or the medical image processing apparatus 30. Since a known configuration can be applied as a configuration of the MRI apparatus 10, description thereof will be omitted. The MRI apparatus 10 is an example of an image capturing apparatus.

It is assumed here that a first medical image and a second medical image are MRI images. The first medical image is collected by, for example, performing predetermined imaging of an imaging site of the subject. Hereinafter, for specific description, it is assumed that the first medical image is a diffusion weighted image (DWI). The diffusion weighted image also includes an apparent diffusion coefficient (ADC) map. The first medical image is assumed to be an axial section of the imaging site or a coronal section of the imaging site. The imaging site is assumed to be the brain of the subject. The predetermined imaging is, for example, diffusion weighted (DW) imaging using echo planar imaging (EPI) (DW-EPI). As the DW imaging, a known imaging method of a T2-weighted image system can be applied, and thus description thereof will be omitted. The DW imaging is typically performed on an axial section of the imaging site or a coronal section of the imaging site.

The second medical image is collected by imaging different from the predetermined imaging, and includes an imaging site related to the collecting of the first medical image. The second medical image is assumed to be a fluid attenuated inversion recovery (FLAIR) image. The second medical image is assumed to be an axial section of the imaging site or a coronal section of the imaging site. The imaging site is assumed to be the brain of the subject as with the first medical image. The imaging different from the predetermined imaging is, for example, FLAIR imaging using a fast spin echo method (FLAIR-FSE). As the FLAIR imaging, a known imaging method of a water-suppressed T2-weighted image system can be applied, and thus description thereof will be omitted. The FLAIR imaging is typically performed on an axial section of the imaging site or a coronal section of the imaging site. The second medical image may be the same section as the first medical image, or may be a section different from the first medical image.

The DWI imaging and the FLAIR imaging may be performed for a volume scan of the brain of the subject or may be performed on other sections, such as an oblique section, without being limited to the axial section of the imaging site or the coronal section of the imaging site. At this time, the first medical image and the second medical image are generated by performing section conversion processing on generated volume data.

The image storage apparatus 20 stores the first medical image and the second medical image collected by the MRI apparatus 10. For example, the image storage apparatus 20 is achieved by computer equipment, such as a server apparatus. Specifically, the image storage apparatus 20 is achieved by a picture archiving and communication system (PACS) server or the like. The image storage apparatus 20 may be referred to as a medical image management system. In the present exemplary embodiment, the image storage apparatus 20 acquires the first medical image and the second medical image from the MRI apparatus 10 via the network, and stores the acquired first medical image and second medical image in a memory provided inside or outside the apparatus.

The medical image processing apparatus 30 acquires the first medical image and the second medical image from the MRI apparatus 10 or the image storage apparatus 20 via the network, and performs various processes using the acquired first medical image and second medical image. The medical image processing apparatus 30 may be referred to as a medical image analysis apparatus or an analysis apparatus. For example, the medical image processing apparatus 30 is achieved by computer equipment, such as a workstation. The medical image processing apparatus 30 causes a display 32 to display a result of processing performed based on the first medical image and the second medical image.

As illustrated in FIG. 1, the medical image processing apparatus 30 includes an input interface 31, the display 32, a memory 33, and processing circuitry 34.

The input interface 31 is achieved by a trackball, a switch, a button, a mouse, a keyboard, a touch pad for performing an input operation by touching an operation surface, a touch screen in which a display screen and a touch pad are integrated, non-contact input circuitry using an optical sensor, voice input circuitry, and the like for issuing various instructions performing various settings, and the like. The input interface 31 converts an input operation received from an operator into an electric signal and outputs the electric signal to the processing circuitry 34. The input interface 31 is not limited to an interface including physical operation components such as a mouse and a keyboard. Examples of the input interface 31 also include electric signal processing circuitry that receives an electric signal corresponding to an input operation from an external input device provided separately from the medical image processing apparatus 30 and outputs the electric signal to the processing circuitry 34. The input interface 31 is an example of an input unit.

The display 32 displays various kinds of information under the control of a display control function 34f. For example, the display 32 displays a graphical user interface (GUI) for receiving an instruction from the operator and various types of X-ray image data. For example, the display 32 is a liquid crystal display or a cathode ray tube (CRT) display. The display 32 is an example of a display unit.

The memory 33 is achieved by, for example, a semiconductor memory element such as a random access memory (RAM) or a flash memory, a hard disk, or an optical disk. For example, the memory 33 stores the first medical image and the second medical image acquired from the MRI apparatus 10 or the image storage apparatus 20. For example, the memory 33 stores a program for each circuitry included in the medical image processing apparatus 30 to achieve its function. The memory 33 is an example of a storage unit.

The processing circuitry 34 controls the overall operation of the medical image processing apparatus 30 by executing an acquisition function 34a, an identification function 34b, a determination function 34c, a decision function 34d, a calculation function 34e, and the display control function 34f.

The processing circuitry 34 acquires the first medical image and the second medical image from the MRI apparatus 10 or the image storage apparatus 20 by reading a program corresponding to the acquisition function 34a from the memory 33 and executing the program. The acquisition function 34a stores the first medical image and the second medical image in the memory 33. The processing circuitry 34 that achieves the acquisition function 34a corresponds to an acquisition unit.

The processing circuitry 34 reads a program corresponding to the identification function 34b from the memory 33 and executes the program. By reading and executing the program, the identification function 34b identifies a first area related to a lesion in the imaging site, based on the first medical image. For example, the identification function 34b executes image processing on the first medical image and identifies the first area in the first medical image. For example, the identification function 34b identifies, as the first area, a plurality of pixels having pixel values lower than a predetermined threshold value in an ADC map of the first medical image. The predetermined threshold value corresponds to, for example, a value (hereinafter, referred to as an ADC threshold value) at which an apparent diffusion coefficient (ADC) is lower than that of a healthy portion. The ADC threshold value is set in advance and stored in the memory 33. A small value of the apparent diffusion coefficient corresponds to a tissue affected by an infarction of a blood vessel. Thus, in a case where the imaging site is a brain, the first area corresponds to an area where a cerebral infarction has occurred.

Identification of the first area is not limited to segmentation processing using the ADC threshold value for the ADC map in the first medical image. For example, a trained model that receives the first medical image as an input and outputs the first area may be used to identify the first area.

The processing circuitry 34 performs alignment (registration) between the first medical image and the second medical image and then identifies positional association between the first medical image and the second medical image, using the identification function 34b. The positional association corresponds to, for example, a transformation matrix (mapping matrix) for associating each of a plurality of pixels in the first medical image with a corresponding one of a plurality of pixels in the second medical image. Since a known method can be applied to the registration and generation of the mapping matrix, description thereof will be omitted. The identification function 34b stores the mapping matrix in the memory 33.

The processing circuitry 34 identifies a center line with respect to the imaging site in the second medical image, using the identification function 34b. In a case where the imaging site is a substantially bilaterally symmetrical organ (i.e., a substantially line-symmetrical organ) such as a brain, the center line corresponds to a symmetry axis regarding substantially bilateral symmetry, i.e., a symmetry axis regarding the substantially line-symmetrical organ. Since a known method can be applied as the image processing for identifying the center line, description thereof will be omitted. The identification function 34b stores the center line (symmetry axis) identified for the imaging site in the second medical image in the memory 33. The processing circuitry 34 that achieves the identification function 34b corresponds to an identification unit.

The processing circuitry 34 determines whether the first area is bilateral or unilateral with respect to the imaging site, based on a position of the first area in the imaging site, using the determination function 34c. The position of the first area in the imaging site corresponds to, for example, the position of the first area with respect to a symmetry axis in a bilaterally symmetrical imaging site. The determination function 34c determines whether the first area is bilateral or unilateral with respect to the imaging site by using the position of the first area in the imaging site in the second medical image. The processing circuitry 34 that achieves the determination function 34c corresponds to a determination unit.

To be specific, the processing circuitry 34 maps the first area on the second medical image using the mapping matrix, using the determination function 34c. Subsequently, the determination function 34c calculates line symmetry of the first area in the second medical image with the center line relative to the imaging site as a symmetry axis in the second medical image, and identifies a symmetric area corresponding to the line symmetry. The determination function 34c determines whether or not the first area and the symmetric area overlap with each other in the second medical image. In a case where the first area and the symmetric area overlap with each other, the determination function 34c determines that the first area is bilateral. In a case where the first area and the symmetric area do not overlap with each other, the determination function 34c determines that the first area is unilateral. In the above description, whether the first area is bilateral or unilateral has been determined in the second medical image, but the present exemplary embodiment is not limited thereto, and whether the first area is bilateral or unilateral may be determined in the first medical image.

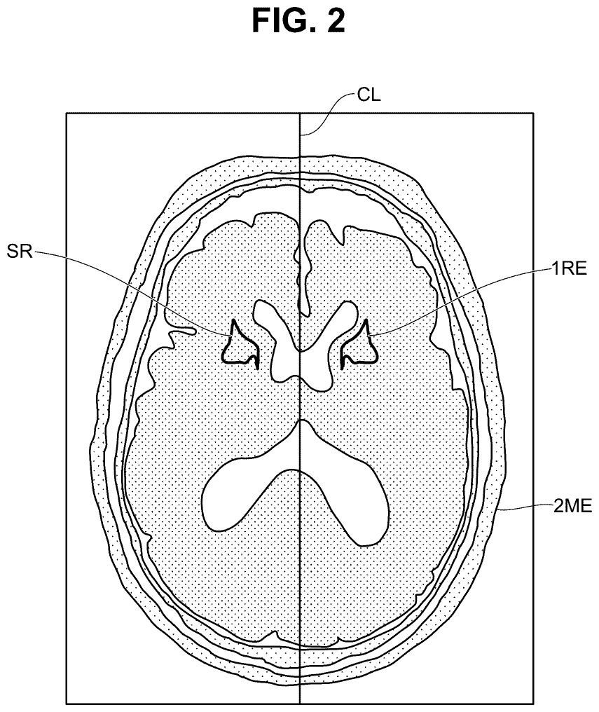

FIG. 2 is a diagram illustrating an example of a first area 1RE and a symmetric area SR in a second medical image 2ME. As illustrated in FIG. 2, the symmetric area SR is set at a position corresponding to line symmetry of the first area 1RE with a center line CL as a symmetry axis. As illustrated in FIG. 2, since the first area 1RE and the symmetric area SR do not overlap with each other, the determination function 34c determines that the first area 1RE is unilateral.

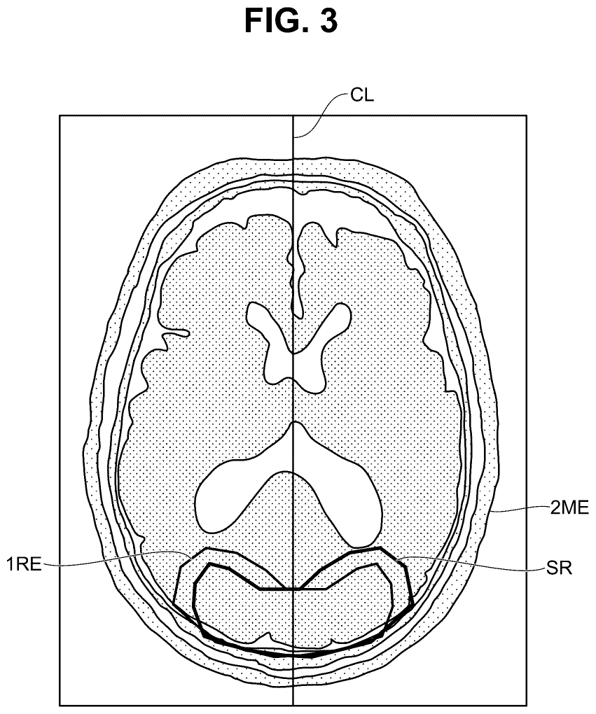

FIG. 3 is a diagram illustrating an example of the first area 1RE and the symmetric area SR in the second medical image 2ME. As illustrated in FIG. 3, the symmetric area SR is set at a position corresponding to line symmetry of the first area 1RE with the center line CL as a symmetry axis, and a part of the symmetric area SR overlaps with the first area 1RE. Therefore, the determination function 34c determines that the first area 1RE is bilateral.

The processing circuitry 34 may determine that the first area 1RE is bilateral in a case where a size of the overlapping area where the symmetric area SR and the first area 1RE overlap with each other exceeds a predetermined threshold value, and may determine that the first area 1RE is unilateral in a case where the size of the overlapping area is less than or equal to the predetermined threshold value, using the determination function 34c. The predetermined threshold value is a threshold value related to a determination of whether the first area 1RE is bilateral or unilateral (hereinafter, referred to as a determination threshold value), for example, a volume, an area, the number of voxels, the number of pixels, or the like set in advance, and the predetermined threshold value is stored in the memory 33.

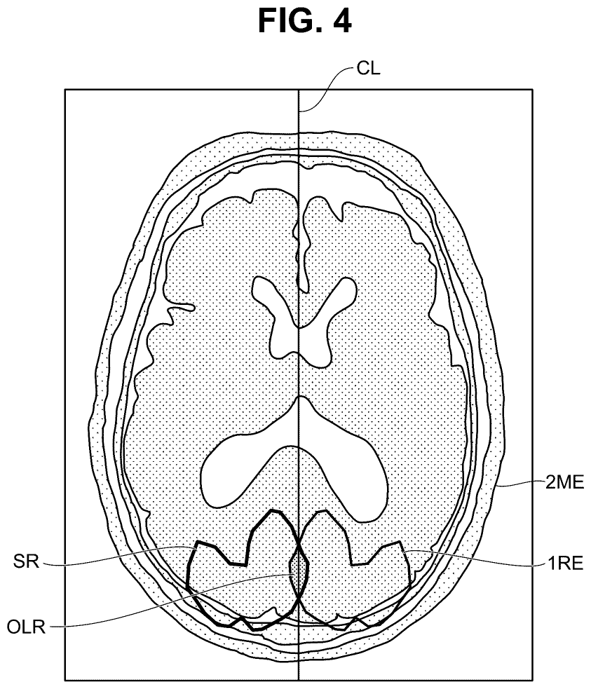

FIG. 4 is a diagram illustrating an example of the first area 1RE and the symmetric area SR in the second medical image 2ME. As illustrated in FIG. 4, the symmetric area SR is set at a position corresponding to line symmetry of the first area 1RE with the center line CL as a symmetry axis, and a part of the symmetric area SR overlaps with the first area 1RE. As a result, as illustrated in FIG. 4, by comparing a size of an overlapping area OLR where the symmetric area SR and the first area 1RE overlap with each other with a determination threshold value, the determination function 34c determines whether the first area 1RE is unilateral or bilateral.

The processing circuitry 34 may determine whether or not the first area 1RE is included in an anatomic site located across the center line CL with respect to the imaging site. The processing circuitry 34 may determine that the first area 1RE is bilateral in a case where the first area 1RE is included in the anatomic site, and may determine that the first area 1RE is unilateral in a case where the first area 1RE is not included in the anatomic site, using the determination function 34c. In a case where the imaging site is the brain, the anatomic site is, for example, a cerebellum, a brain stem, or the like.

To be specific, prior to the determination by the determination function 34c, the processing circuitry 34 identifies the anatomic site by performing predetermined image processing on the second medical image, using the identification function 34b. As the predetermined image processing, for example, segmentation processing such as brain parcellation and a known method such as a trained model can be applied, and thus description thereof will be omitted. The brain parcellation corresponds to, for example, an area division of the brain according to the function of the brain. At this time, the identification function 34b identifies a plurality of segmented areas corresponding to the functions of the brain by performing the brain parcellation on the image of the brain. Then, the determination function 34c compares the first area 1RE to which the mapping matrix has been applied with the identified anatomic site, determines that the first area 1RE is bilateral in a case where the first area 1RE is included in the anatomic site, and determines that the first area 1RE is unilateral in a case where the first area 1RE is not included in the anatomic site.

The processing circuitry 34 decides a second area 2RE indicating a normal portion in the imaging site, using the decision function 34d. The second area 2RE is an area different from the first area 1RE in the second medical image 2ME, and corresponds to a normal area indicating a normal tissue. For example, in a case where it is determined that the first area 1RE is unilateral, the decision function 34d decides, as the second area 2RE, an area corresponding to line symmetry of the first area 1RE with the center line CL relative to the imaging site as a symmetry axis in the second medical image 2ME. As illustrated in FIG. 2, in a case where the first area 1RE is unilateral, the second area 2RE corresponds to the symmetric area SR. The processing circuitry 34 that achieves the decision function 34d corresponds to a decision unit.

The decision function 34d may decide the entire area other than the first area 1RE in the second medical image 2ME as the second area 2RE. At this time, for example, the decision function 34d decides the second area 2RE by subtracting the first area 1RE from the second medical image 2ME. The decision function 34d may decide the second area 2RE by inputting the second medical image 2ME and the first area 1RE to a trained model capable of outputting the second area 2RE.

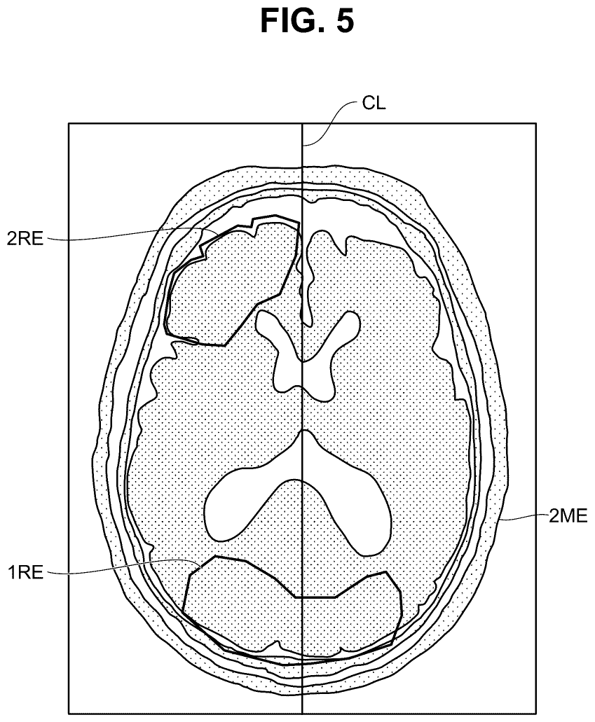

In a case where it is determined that the first area 1RE is bilateral, the processing circuitry 34 decides an area different from the first area 1RE as the second area 2RE in the second medical image, using the decision function 34d. FIG. 5 is a diagram illustrating an example of the first area 1RE and the second area 2RE in a case where the first area 1RE is bilateral. A position and a size of the second area 2RE with respect to the imaging site are not limited to those illustrated in FIG. 5, and can be arbitrarily set.

For example, in a case where it is determined that the first area 1RE is bilateral, the processing circuitry 34 may decide, as the second area 2RE, an area having an apparent diffusion coefficient (ADC value) exceeding a predetermined value in the second medical image, using the decision function 34d. The predetermined value is, for example, an ADC threshold value. The predetermined value may be set to a value greater than the ADC threshold value. Specifically, the decision function 34d decides an area exceeding the predetermined value (hereinafter, referred to as an excess area) in the ADC map. Next, the decision function 34d applies the mapping matrix to the excess area and maps the excess area onto the second medical image 2ME. Subsequently, the decision function 34d decides, as the second area 2RE, an area different from the first area 1RE in the mapped excess area in the second medical image.

The processing circuitry 34 may apply the mapping matrix to the ADC map and map the ADC map onto the second medical image 2ME, and then apply a predetermined value to the ADC map mapped onto the second medical image 2ME to decide the excess area, using the decision function 34d. At this time, the decision function 34d decides, as the second area 2RE, an area different from the first area 1RE in the determined excess area in the second medical image.

In a case where it is determined that the first area 1RE is bilateral, the processing circuitry 34 may decide, as the second area 2RE, an area that does not include cerebral spinal fluid of the subject and an area on the outside of the brain (i.e., an area where the imaging site is not displayed) in the area different from the first area 1RE in the second medical image, using the decision function 34d. Specifically, prior to the decision of the second area 2RE by the decision function 34d, the identification function 34b identifies an area of the cerebral spinal fluid and an area on the outside of the brain by performing predetermined image processing on the second medical image. As the predetermined image processing, known methods such as various kinds of segmentation processing and a trained model can be applied, and thus description thereof will be omitted. Next, the decision function 34d decides, as the second area 2RE, an area that does not include the area of the cerebral spinal fluid of the subject and the area on the outside of the brain and that is different from the first area 1RE in the second medical image.

In a case where it is determined that the first area 1RE is bilateral, the processing circuitry 34 may decide, as the second area 2RE, an excess area that does not include the area of the cerebral spinal fluid of the subject and the area on the outside of the brain and that exceeds the predetermined value in the ADC map to which the mapping matrix has been applied, in the area different from the first area 1RE in the second medical image, using the decision function 34d.

In a case where it is determined that the first area 1RE is bilateral, the processing circuitry 34 may decide, as the second area 2RE, an area similar to the property of a tissue in the first area 1RE in the second medical image, using the decision function 34d. For example, prior to the decision of the second area 2RE by the decision function 34d, the identification function 34b identifies an area similar to the first area 1RE in tissue property (hereinafter, referred to as a similar area) by performing predetermined image processing on the second medical image. As the predetermined image processing, segmentation processing such as brain parcellation and a known method such as a trained model can be applied, and thus description thereof will be omitted. Next, the decision function 34d decides, as the second area 2RE, a similar area in an area different from the first area 1RE in the second medical image.

In a case where it is determined that the first area 1RE is bilateral, the processing circuitry 34 may determine, as the second area, only a specific tissue, such as white matter or gray matter, in the area different from the first area 1RE in the second medical image 2ME, using the decision function 34d. For example, in a case where the first area 1RE is a cerebellar infarction, the decision function 34d may decide a non-cerebellum area in the second medical image as the second area. In order to identify the specific tissue, such as white matter or gray matter, and an area other than the cerebellum, segmentation processing, such as brain parcellation to be performed on the second medical image, and a known method, such as a trained model, can be applied, and thus description thereof will be omitted. For example, the decision function 34d may divide the brain area in the second medical image using an automatic brain area extraction application or the like, and select the second area in the second medical image.

The processing circuitry 34 calculates, using the calculation function 34e, a first feature amount indicating a feature that is based on a plurality of pixel values in the first area 1RE and a second feature amount indicating a feature that is based on a plurality of pixel values in the second area 2RE in the second medical image 2ME. The first feature amount is a statistical value based on a plurality of pixel values included in the first area 1RE in the second medical image 2ME. The second feature amount is a statistical value based on a plurality of pixel values included in the second area 2RE in the second medical image 2ME. The statistical value is, for example, a median value of a plurality of pixel values. In other words, the first feature amount is a median value (hereinafter, referred to as a first median value) of the plurality of pixel values included in the first area 1RE in the second medical image 2ME. The second feature amount is a median value (hereinafter, referred to as a second median value) of the plurality of pixel values included in the second area 2RE in the second medical image 2ME. The statistical value corresponding to the second feature amount may be a standard deviation (hereinafter referred to as a normal standard deviation) of the plurality of pixel values in the second area 2RE. The statistical value is not limited to the above, and a known statistical value can be appropriately used.

The processing circuitry 34 calculates an index related to a mismatch (hereinafter referred to as a mismatch index) between the first medical image and the second medical image, based on the first feature amount and the second feature amount, using the calculation function 34e. The mismatch index is, for example, a ratio between the first feature amount and the second feature amount. To be specific, the calculation function 34e calculates a ratio of the first median value to the second median value as the mismatch index. The calculation function 34e may calculate a ratio of each of the plurality of pixel values in the first area 1RE to the second median value as a mismatch index map in the first area 1RE. In a case where the second median value is set to 0, the calculation function 34e may calculate, as the mismatch index map in the first area 1RE, a value indicating how many times greater each of the plurality of pixel values in the first area 1RE is than the normal standard deviation. At this time, each value (mismatch index) of the mismatch index map is equivalent to a numerical value corresponding to a Z-score.

The processing circuitry 34 stores the mismatch index or the mismatch index map calculated by the calculation function 34e in the memory 33 in association with the first area 1RE. The processing circuitry 34 that achieves the calculation function 34e corresponds to a calculation unit.

The processing circuitry 34 reads a program corresponding to the display control function 34f from the memory 33 and executes the program. As a result, the display control function 34f causes the display 32 to display the mismatch index, which is calculated by the calculation function 34e, the first area 1RE, and the second area 2RE. For example, the display control function 34f causes the display 32 to display a superimposed image in which the first area 1RE, the second area 2RE, and the mismatch index are superimposed on the second medical image. At this time, the display 32 displays the first area 1RE and the second area 2RE superimposed on the second medical image 2ME together with the mismatch index. The processing circuitry 34 that achieves the display control function 34f corresponds to a display control unit.

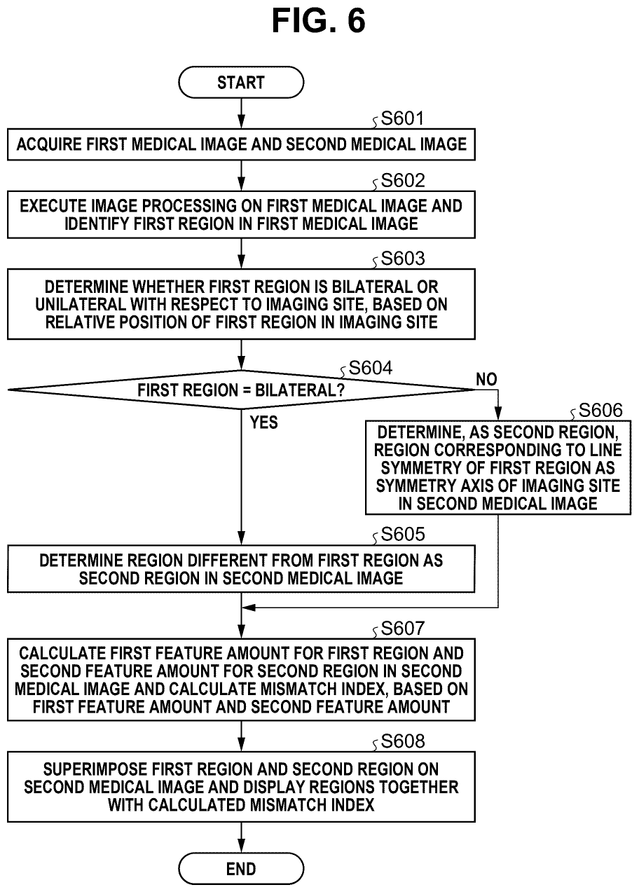

The overall configuration of the medical information processing system 1 according to the exemplary embodiment has been described above. Hereinafter, processing related to quantitative evaluation of a mismatch between the first medical image and the second medical image (hereinafter, referred to as mismatch evaluation processing) will be described with reference to FIG. 6.

FIG. 6 is a flowchart illustrating an example of a procedure of mismatch evaluation processing. Hereinafter, in order to make the description specific, it is assumed that the imaging site is the brain of the subject as described above, the first medical image is a diffusion weighted image, and the second medical image is a FLAIR image. It is assumed that the first medical image and the second medical image are generated in advance before the mismatch evaluation processing is performed.

### (Mismatch Evaluation Processing)

### (Step S601)

The processing circuitry 34 acquires the first medical image and the second medical image from the MRI apparatus 10 or the image storage apparatus 20, using the acquisition function 34a. The acquisition function 34a stores the first medical image and the second medical image in the memory 33.

### (Step S602)

The processing circuitry 34 executes image processing on the first medical image and identifies the first area in the first medical image, using the identification function 34b. For example, the identification function 34b identifies, as the first area, a plurality of pixels having respective pixel values lower than ADC threshold value in the ADC map of the first medical image. The identification function 34b stores the first area 1RE identified on the second medical image by the mapping matrix in the memory 33.

### (Step S603)

The processing circuitry 34 determines whether the first area is bilateral or unilateral with respect to the imaging site, based on a position of the first area in the imaging site, using the determination function 34c. For example, in a case where the first area lies while crossing over a symmetry axis (center line) in the second medical image, the determination function 34c determines that the first area is bilateral. Other determination methods conform to the above description, and thus description thereof will be omitted.

### (Step S604)

In a case where the first area is determined to be bilateral (Yes in step S604), the processing in step S605 is performed. In a case where the first area 1RE is determined to be not bilateral (No in step S604), i.e., in a case where it is determined that the first area is unilateral, processing in step S606 is performed.

### (Step S605)

The processing circuitry 34 decides an area different from the first area 1RE as the second area 2RE in the second medical image 2ME, using the decision function 34d. A method of deciding the second area 2RE in a case where the first area is determined to be bilateral conforms to the above description, and thus description thereof will be omitted. The decision function 34d stores the second area 2RE decided on the second medical image in the memory 33.

### (Step S606)

The processing circuitry 34 decides, as the second area 2RE, an area (target area) corresponding to line symmetry of the first area 1RE with a symmetry axis of the imaging site in the second medical image 2ME, using the decision function 34d. A method of deciding the second area 2RE in a case where the first area 1RE is determined to be unilateral conforms to the above description, and thus description thereof will be omitted. The decision function 34d stores the second area 2RE decided on the second medical image in the memory 33.

### (Step S607)

The processing circuitry 34 calculates, using the calculation function 34e, a first feature amount for the first area 1RE and a second feature amount for the second area 2RE in the second medical image 2ME. Since processing details related to the calculation of the first feature amount and the second feature amount conform to the description provided above, description thereof will be omitted. The calculation function 34e calculates a mismatch index based on the first feature amount and the second feature amount. Processing details related to the calculation of the mismatch index conform to the description provided above, and description thereof will be omitted. The calculation function 34e stores the mismatch index in the memory 33 in association with the first area 1RE and the second area 2RE.

### (Step S608)

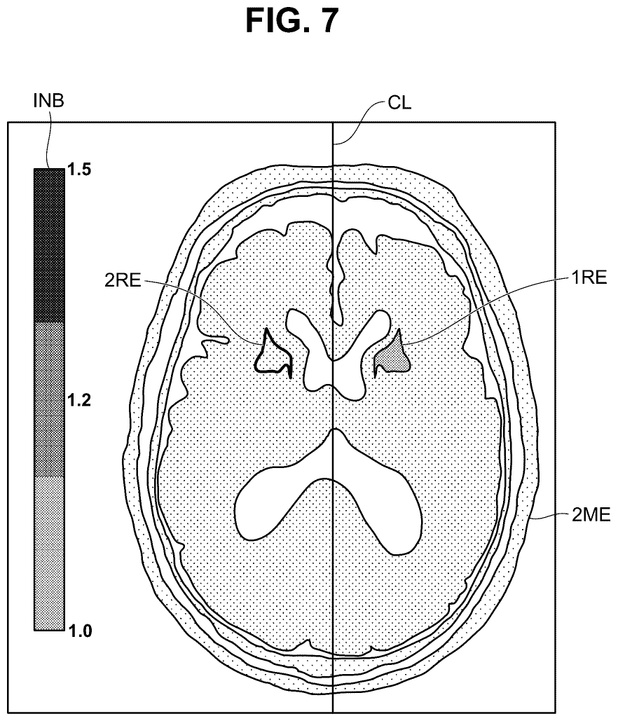

The processing circuitry 34 superimposes the first area 1RE and the second area 2RE on the second medical image 2ME and causes the display 32 to display the first area 1RE and the second area 2RE together with the calculated mismatch index, using the display control function 34f. Hereinafter, a display example of a case where the first area 1RE is unilateral and a display example of a case where the first area 1RE is bilateral will be described with reference to FIGS. 7 to 9.

FIG. 7 is a diagram illustrating an example in which the first area 1RE and the second area 2RE are superimposed on the second medical image 2ME and displayed on the display 32 together with a legend INB of mismatch indices in a case where the first area 1RE is determined to be unilateral. In FIG. 7, a ratio of a first median value to a second median value is superimposed on the first area 1RE and displayed as the mismatch index on the display 32.

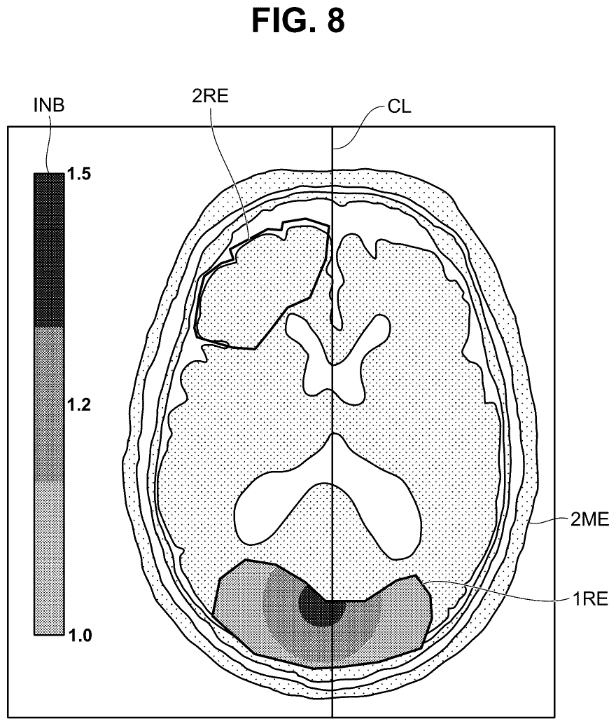

FIG. 8 is a diagram illustrating an example in which the first area 1RE and the second area 2RE are superimposed on the second medical image 2ME and are displayed on the display 32 together with the legend INB of mismatch indices in a case where the first area 1RE is determined to be bilateral. In FIG. 8, the ratio of the first median value to the second median value is superimposed on the first area 1RE and displayed as the mismatch index on the display 32.

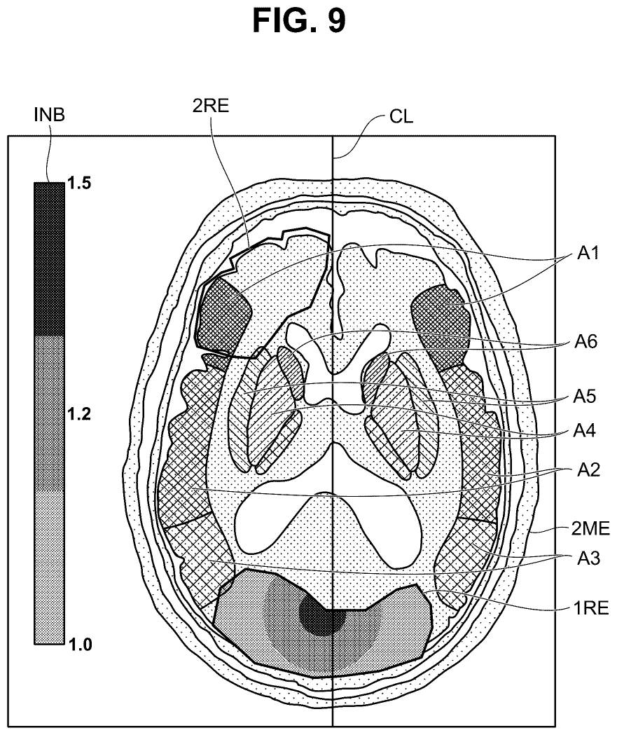

FIG. 9 is a diagram illustrating an example in which a plurality of segment areas is further superimposed in the display example illustrated in FIG. 8. For example, in FIG. 9, the plurality of segment areas (A1 to A6) is superimposed on the second medical image 2ME, and then the first area 1RE and the second area 2RE are superimposed thereon. As illustrated in FIG. 9, by superimposing the plurality of segment areas corresponding to the functions of the brain, it is possible to easily understand the correspondence between functions of the brain in a lesion area and a normal area. Therefore, based on the display example illustrated in FIG. 9, since functional areas of the brain related to the lesion area and the normal area can be easily understood, it is possible to reduce a burden of examination for the user. Based on the above, the medical image processing apparatus 30 according to the exemplary embodiment can improve a throughput of the examination on the subject.

The medical image processing apparatus 30 according to the exemplary embodiment described above acquires the first medical image and the second medical image 2ME, identifies the first area 1RE based on the first medical image, determines whether the first area 1RE is bilateral or unilateral based on a position of the first area 1RE in the imaging site in the second medical image 2ME, decides, as the second area 2RE, an area different from the first area 1RE in the second medical image 2ME in a case where it is determined that the first area 1RE is bilateral, decides, as the second area 2RE, an area corresponding to line symmetry of the first area 1RE with a center line CL relative to the imaging site as a symmetry axis in the second medical image 2ME in a case where it is determined that the first area 1RE is unilateral, calculates a first feature amount related to the first area 1RE and a second feature amount related to the second area 2RE in the second medical image, and calculates a mismatch index based on the first feature amount and the second feature amount. The medical image processing apparatus 30 according to the exemplary embodiment superimposes the first area 1RE and the second area 2RE on the second medical image 2ME and displays the first area 1RE and the second area 2RE together with the mismatch index.

In the medical image processing apparatus 30 according to the exemplary embodiment, the first medical image and the second medical image are axial sections of the imaging site or coronal sections of the imaging site. In the medical image processing apparatus 30 according to the exemplary embodiment, the first medical image is a diffusion weighted image, and the second medical image is a FLAIR image. In the medical image processing apparatus 30 according to the exemplary embodiment, the first feature amount is a statistical value based on a plurality of pixel values included in the first area 1RE, and the second feature amount is a statistical value based on a plurality of pixel values included in the second area 2RE. In the medical image processing apparatus 30 according to the exemplary embodiment, the mismatch index is a ratio between the first feature amount and the second feature amount.

The medical image processing apparatus 30 according to the exemplary embodiment may determine that the first area 1RE is bilateral in a case where the first area 1RE overlaps with a symmetric area SR corresponding to line symmetry of the first area 1RE with a center line relative to the imaging site as a symmetry axis, and may determine that the first area 1RE is unilateral in a case where the first area 1RE does not overlap with the symmetric area SR. The medical image processing apparatus 30 according to the exemplary embodiment may determine that the first area 1RE is bilateral in a case where a size of the overlapping area OLR in which the symmetric area SR and the first area 1RE overlap with each other exceeds a predetermined threshold value, and may determine that the first area 1RE is unilateral in a case where the size of the overlapping area OLR is less than or equal to the predetermined threshold value.

The medical image processing apparatus 30 according to the exemplary embodiment may identify an anatomic site located across the center line CL relative to the imaging site by predetermined image processing on the second medical image 2ME, determine whether or not the first area 1RE is included in the identified anatomic site, determine that the first area 1RE is bilateral in a case where the first area 1RE is included in the anatomic site, and determine that the first area 1RE is unilateral in a case where the first area 1RE is not included in the anatomic site.

In a case where it is determined that the first area 1RE is bilateral, the medical image processing apparatus 30 according to the exemplary embodiment may decide an area having an apparent diffusion coefficient exceeding a predetermined value as the second area 2RE. In the medical image processing apparatus 30 according to the exemplary embodiment, the imaging site is the brain of the subject, and in a case where it is determined that the first area 1RE is bilateral, the medical image processing apparatus 30 according to the exemplary embodiment may decide an area that does not include the cerebral spinal fluid of the subject and an area outside the brain as the second area 2RE. In a case where it is determined that the first area 1RE is bilateral, the medical image processing apparatus 30 according to the exemplary embodiment may decide an area similar to the property of tissue in the first area 1RE as the second area 2RE.

Based on the above, the medical image processing apparatus 30 according to the exemplary embodiment can determine whether a lesion (for example, a cerebral infarction) of a subject is unilateral or bilateral, and appropriately set an area on the unaffected side (normal area) serving as a reference for calculation of a mismatch index to determine whether or not a lesion area is in the acute phase. Accordingly, the medical image processing apparatus 30 according to the exemplary embodiment can improve accuracy of quantitative evaluation of a DWI/FLAIR mismatch phenomenon regardless of whether the lesion is bilateral or unilateral. In addition, the medical image processing apparatus 30 according to the exemplary embodiment does not require a doctor, i.e., a user, to qualitatively determine the DWI/FLAIR mismatch phenomenon in his or her own head, and thus can reduce a burden of examination for the user. Furthermore, the medical image processing apparatus 30 according to the exemplary embodiment can reduce the qualitative determinations to be made by the user, and therefore can improve the throughput of the examination on the subject.

### Application Example

In this application example, it is determined whether or not a lesion is in the acute phase based on a mismatch index, and a determination result is displayed on the display 32. The memory 33 stores a threshold value for determining whether or not the lesion is in the acute phase (hereinafter referred to as an acute phase determination threshold value). In mismatch evaluation processing in this application example, the following processing is executed after step S607 in FIG. 6. The processing circuitry 34 compares the mismatch index with the acute phase determination threshold value, using the determination function 34c. In a case where the mismatch index exceeds the acute phase determination threshold value, the determination function 34c determines that a lesion related to the first area 1RE is in the acute phase.

In the mismatch evaluation processing in the application example, the following processing is further executed in step S608 in FIG. 6. The processing circuitry 34 causes the display 32 to display the mismatch index and the determination result as to whether or not the lesion is in the acute phase, together with a superimposed image acquired by superimposing the first area 1RE and the second area 2RE on the second medical image 2ME, using the display control function 34f.

As a modification example of this application example, the memory 33 may store an algorithm (hereinafter, referred to as a lesion onset calculation algorithm) such as a calculation formula or a trained model for calculating an onset time of a lesion related to the first area 1RE and/or an elapsed time from the onset of the lesion using the mismatch index as an input. At this time, the processing circuitry 34 calculates the onset time of the lesion and/or the elapsed time from the onset of the lesion by applying the mismatch index to the lesion onset calculation algorithm, using the calculation function 34e. Next, the processing circuitry 34 causes the display 32 to display the onset time of the lesion and/or the elapsed time from the onset of the lesion, in addition to the superimposed image acquired by superimposing the first area 1RE and the second area 2RE on the second medical image 2ME, the mismatch index, and the determination result as to whether or not the lesion is in the acute phase, using the display control function 34f.

Instead of the lesion onset calculation algorithm, a correspondence table of the onset time of the lesion and/or the elapsed time from the onset of the lesion with respect to the mismatch index may be stored in the memory 33. At this time, the processing circuitry 34 decides the onset time of the lesion and/or the elapsed time from the onset of the lesion by collating the mismatch index with the correspondence table, using the decision function 34d.

The medical image processing apparatus 30 according to this application example can predict the onset time and the like of a unilateral and bilateral lesion (for example, cerebral infarction). Thus, the medical image processing apparatus 30 according to this application example can further reduce the burden of examination for the user and further improve a throughput of the examination on the subject. Since other effects are the same as those of the exemplary embodiment, description thereof will be omitted.

In a case where the technical idea of the present exemplary embodiment is achieved by a medical image processing method, the medical image processing method includes acquiring a first medical image collected by predetermined imaging on an imaging site of a subject and a second medical image 2ME collected by imaging different from the predetermined imaging and including the imaging site, identifying a first area 1RE related to a lesion in the imaging site based on the first medical image, determining whether the first area 1RE is bilateral or unilateral with respect to the imaging site based on a position of the first area 1RE in the imaging site, deciding an area different from the first area 1RE in the second medical image 2ME as a second area 2RE indicating a normal portion in the imaging site in a case where it is determined that the first area 1RE is bilateral, deciding, as the second area 2RE, an area corresponding to line symmetry of the first area 1RE with a center line CL relative to the imaging site as a symmetry axis in the second medical image 2ME in a case where it is determined that the first area 1RE is unilateral, calculating a first feature amount indicating a feature that is based on a plurality of pixel values in the first area 1RE and a second feature amount indicating a feature that is based on a plurality of pixel values in the second area 2RE in the second medical image 2ME, and calculating an index related to a mismatch between the first medical image and the second medical image 2ME based on the first feature amount and the second feature amount. A processing procedure in the medical image processing method conforms to the procedure of the mismatch evaluation processing. An effect of the medical image processing method is the same as that of the exemplary embodiment. For these reasons, descriptions of the processing procedure and the effect of the mismatch evaluation processing in the medical image processing method will be omitted.

In a case where the technical idea in the exemplary embodiment is achieved by a medical image processing program, the medical image processing program causes a computer to acquire a first medical image collected by predetermined imaging on an imaging site of a subject and a second medical image 2ME collected by imaging different from the predetermined imaging and including the imaging site, identify a first area 1RE related to a lesion in the imaging site based on the first medical image, determine whether the first area 1RE is bilateral or unilateral with respect to the imaging site based on a position of the first area 1RE in the imaging site, decide an area different from the first area 1RE in the second medical image 2ME as a second area 2RE indicating a normal portion in the imaging site in a case where it is determined that the first area 1RE is bilateral, decide, as the second area 2RE, an area corresponding to line symmetry of the first area 1RE with a center line CL relative to the imaging site as a symmetry axis in the second medical image 2ME in a case where it is determined that the first area 1RE is unilateral, calculate a first feature amount indicating a feature that is based on a plurality of pixel values in the first area 1RE and a second feature amount indicating a feature that is based on a plurality of pixel values in the second area 2RE in the second medical image 2ME, and calculate an index related to a mismatch between the first medical image and the second medical image 2ME, based on the first feature amount and the second feature amount.

For example, the mismatch evaluation processing can also be achieved by installing an image processing program in a computer, such as the medical image processing apparatus 30 or the MRI apparatus 10 illustrated in FIG. 1, and loading the program on a memory. At this time, the program that can cause the computer to execute the processing can be stored in a storage medium, such as a magnetic disk (hard disk, etc.), an optical disk (CD-ROM, DVD, etc.), or a semiconductor memory and distributed. The distribution of the medical image processing program is not limited to the above-described medium, and for example, the medical image processing program may be distributed by using a telecommunication function, such as downloading via the Internet. The processing procedure in the medical image processing program conforms to the mismatch evaluation processing. An effect of the medical image processing program is the same as that of the exemplary embodiment. For these reasons, descriptions of the processing procedure and the effect of the mismatch evaluation processing in the medical image processing program will be omitted.

The technical features of the present exemplary embodiment can be achieved by an MRI apparatus. In this case, the processing circuitry mounted on the MRI apparatus includes the acquisition function 34a, the identification function 34b, the determination function 34c, the decision function 34d, the calculation function 34e, and the display control function 34f illustrated in FIG. 1. At this time, the MRI apparatus achieves the mismatch evaluation processing. A processing procedure in the MRI apparatus for achieving the acquisition function 34a, the identification function 34b, the determination function 34c, the decision function 34d, the calculation function 34e, and the display control function 34f conforms to the mismatch evaluation processing of the exemplary embodiment. An effect of the MRI apparatus is the same as that of the exemplary embodiment. Therefore, descriptions of the processing procedure and the effect of the mismatch evaluation processing in the MRI apparatus will be omitted.

According to at least the exemplary embodiment, the application example, and the like described above, it is possible to improve the accuracy of quantitative evaluation of mismatch between two medical images regardless of whether a lesion is bilateral or unilateral.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus comprising processing circuitry configured to:
acquire a first medical image collected by predetermined imaging on an imaging site of a subject and a second medical image collected by imaging different from the predetermined imaging and including the imaging site;
identify a first area related to a lesion in the imaging site, based on the first medical image;
determine whether the first area is bilateral or unilateral with respect to the imaging site, based on a position of the first area in the imaging site;
decide an area different from the first area in the second medical image as a second area indicating a normal portion in the imaging site in a case where it is determined that the first area is bilateral;
decide, as the second area, an area corresponding to line symmetry of the first area with a center line relative to the imaging site as a symmetry axis in the second medical image in a case where it is determined that the first area is unilateral; and
calculate, in the second medical image, a first feature amount indicating a feature based on a plurality of pixel values in the first area and a second feature amount indicating a feature based on a plurality of pixel values in the second area, and calculate an index related to a mismatch between the first medical image and the second medical image, based on the first feature amount and the second feature amount.

2. The medical image processing apparatus according to claim 1,
wherein the first medical image and the second medical image are axial sections of the imaging site or coronal sections of the imaging site.

3. The medical image processing apparatus according to claim 1,
wherein the first medical image is a diffusion weighted image, and
wherein the second medical image is a fluid attenuated inversion recovery (FLAIR) image.

4. The medical image processing apparatus according to claim 1,
wherein the first feature amount is a statistical value based on a plurality of pixel values included in the first area, and
wherein the second feature amount is a statistical value based on a plurality of pixel values included in the second area.

5. The medical image processing apparatus according to claim 1, wherein the index is a ratio between the first feature amount and the second feature amount.

6. The medical image processing apparatus according to claim 1,
wherein the processing circuitry determines that the first area is bilateral in a case where the first area overlaps with a symmetric area corresponding to line symmetry of the first area with a center line relative to the imaging site as a symmetry axis, and determines that the first area is unilateral in a case where the first area does not overlap with the symmetric area.

7. The medical image processing apparatus according to claim 1,
wherein the processing circuitry determines that the first area is bilateral in a case where a size of an overlapping area where the first area overlaps with a symmetric area corresponding to line symmetry of the first area with a center line relative to the imaging site as a symmetry axis exceeds a predetermined threshold value, and determines that the first area is unilateral in a case where the size of the overlapping area is less than or equal to the predetermined threshold value.

8. The medical image processing apparatus according to claim 1,
wherein the processing circuitry determines whether or not the first area is included in an anatomic site located across a center line relative to the imaging site, and determines that the first area is bilateral when the first area is included in the anatomic site, and determines that the first area is unilateral in a case where the first area is not included in the anatomic site.

9. The medical image processing apparatus according to claim 8,
wherein the processing circuitry identifies the anatomic site by performing predetermined image processing on the second medical image.

10. The medical image processing apparatus according to claim 1,
wherein, in a case where it is determined that the first area is bilateral, the processing circuitry decides an area having an apparent diffusion coefficient that exceeds a predetermined value as the second area.

11. The medical image processing apparatus according to claim 1,
wherein the imaging site is a brain of the subject, and
wherein, in a case where it is determined that the first area is bilateral, the processing circuitry decides, as the second area, an area that does not include cerebral spinal fluid of the subject and an area outside the brain.

12. The medical image processing apparatus according to claim 1,
wherein, in a case where the first area is determined to be bilateral, the processing circuitry decides an area similar to a property of tissue in the first area as the second area.

13. The medical image processing apparatus according to claim 1, further comprising a display configured to superimpose the first area and the second area on the second medical image together with the index.

14. A medical image processing method comprising:
acquiring a first medical image collected by predetermined imaging on an imaging site of a subject and a second medical image collected by imaging different from the predetermined imaging and including the imaging site;
identifying a first area related to a lesion in the imaging site, based on the first medical image;
determining whether the first area is bilateral or unilateral with respect to the imaging site, based on a position of the first area in the imaging site;
deciding an area different from the first area in the second medical image as a second area indicating a normal portion in the imaging site in a case where it is determined that the first area is bilateral;
deciding, as the second area, an area corresponding to line symmetry of the first area with a center line relative to the imaging site as a symmetry axis in the second medical image in a case where it is determined that the first area is unilateral;
calculating, in the second medical image, a first feature amount indicating a feature based on a plurality of pixel values in the first area and a second feature amount indicating a feature based on a plurality of pixel values in the second area; and
calculating an index related to a mismatch between the first medical image and the second medical image, based on the first feature amount and the second feature amount.

15. A non-transitory computer-readable storage medium storing a medical image processing program for causing a computer to:
acquire a first medical image collected by predetermined imaging on an imaging site of a subject and a second medical image collected by imaging different from the predetermined imaging and including the imaging site;
identify a first area related to a lesion in the imaging site, based on the first medical image;
determine whether the first area is bilateral or unilateral with respect to the imaging site, based on a position of the first area in the imaging site;
decide an area different from the first area in the second medical image as a second area indicating a normal portion in the imaging site in a case where it is determined that the first area is bilateral;
decide, as the second area, an area corresponding to line symmetry of the first area with a center line relative to the imaging site as a symmetry axis in the second medical image in a case where it is determined that the first area is unilateral;
calculate, in the second medical image, a first feature amount indicating a feature based on a plurality of pixel values in the first area and a second feature amount indicating a feature based on a plurality of pixel values in the second area; and
calculate an index related to a mismatch between the first medical image and the second medical image, based on the first feature amount and the second feature amount.
